(19) United States
(12) Patent Application Publication
(10) Pub. No.: US 2008/0154334 A1
(43) Pub. Date: Jun. 26, 2008

(54) Method of pain relief in patients after abdominal/pelvic surgery

(76) Inventor: Stas Gavronsky, Wayland, MA (US)

Correspondence Address: Stas Gavronsky, 39 Wayland Hills Road, Wayland, MA, 01778, US

(21) Appl. No.: 11/645,281

(22) Filed: Dec. 26, 2006

(51) Int. Cl. A61N 1/00

(52) U.S. Cl. 607/46

(57) ABSTRACT

The method of reducing pain in a patient by combining a traditional pharmacological treatment with electro-acupuncture or percutaneous electrical nerve stimulation therapy. The pharmacological treatment consists of using analgesics such as morphine sulfate and hydromorphine provided to patients around the clock or as needed for pain. The patient controlled analgesia pump (PCA) is used by patients for self-administering of analgesia as needed. After patient stops using PCA pump narcotic and non-narcotic pain medications are administered mostly orally. Typically, there are two pairs of needle electrodes, two on each leg, connected to two separate outputs of the portable electrical stimulator. The constructions of the needle holder and the stimulator allow a patient to move freely and, if necessary, to carry the device during the entire day.

### CROSS-REFERENCE TO RELATED APPLICATIONS

The present patent application is related to pending U.S. patent application Ser. No. 11/078,765 filed by the same applicant on Mar. 14, 2005 and entitled “Device for Percutaneous Nerve Stimulation”

### FEDERALLY SPONSORED RESEARCH

(not applicable)

### SEQUENCE LISTING OR PROGRAM

(not applicable)

### FIELD OF THE INVENTION

The present invention relates to the field of electrical therapy for treating pain and other conditions, in particular to pain relief in women after abdominal/pelvic surgery by combining patient-controlled analgesia with percutaneous electrical nerve stimulation therapy. The invention relates to the aforementioned method where percutaneous electrical nerve stimulation therapy or electro-acupuncture are carried out with the use of apparatuses as disclosed in pending U.S. patent application Ser. No. 10/962,299 and in pending U.S. patent application Ser. No. 11/078,765 that allow a patient to have freedom of movements so that the patient can carry the electrodes or needles for the entire day, if needed.

### BACKGROUND OF THE INVENTION

Postoperative pain control represents a major concern for women who undergo gynecological surgery as well as their doctors and nurses. It is essential for providers and nurses to assess, monitor, and provide pharmacologic and nonpharmacologic interventions for those who complain of pain or discomfort, so that the patient will return to self-care and normal daily functioning in a reasonable amount of time. Patient outcome studies indicate that patients accept pain as part of hospitalization (Sherwood, McNeill, Starck, Disnard, 2003). The majority of people who report pain are those who are recovering from surgical intervention. Pain relief and the patient's satisfaction with care frequently are used to measure pain management outcomes (Sherwood, et. al., 2003). Pain relief is evaluated by patient self-report of pain intensity, worst pain, interference with activities, and overall pain (Sherwood, et. al., 2003). Patient satisfaction is an important but elusive outcome indicator, and informed patients may choose other providers if their expectations are not met (Sherwood, et. al., 2003). Pain relief and patient satisfaction are considered subjective assessments, but are also fairly good indicators of quality of care. Patients who experience continuous pain can result in morbidity as well as delayed discharge. Postoperative pain evokes both physiological changes and psychological responses, which suggests that a combination of pharmacological and nonpharmacological approaches can enhance the effect of pain relieving medication (Sherwood, et. al., 2003). An adjunctive approach can help patients feel a sense of control over pain, which can also influence the patient's satisfaction.

For some time now analgesic interventions have been the primary source of providing pain relief. Analgesics such as morphine sulfate and hydromorphine have been provided to patients around the clock or as needed for pain. The patient controlled analgesia pump (PCA) is also another way to examine the pattern of patient attempts at self-administration of analgesia so that nurses and providers can figure out the patient's pattern of pain perception. The PCA pump provides a method of identifying individual pain rhythm and assessing individual analgesic needs (Sherwood, et. al., 2003). This infusion device is connected to an intravenous, subcutaneous, ventricular, epidural, or subarachnoid catheter, and narcotic analgesics can be administered by the patient's activating a button attached to the pump (William, 1992). This device can be programmed to deliver a specified dose of medication on demand at predetermined time intervals (usually every 5 to 10 minutes) and helps prevent the patient from overdosing or abusing the analgesics (William, 1992). PCA pumps allow the patient to administer a dose of analgesic at the onset of pain sensation. This results in immediate relief. This method maintains minimum effective analgesic serum concentration without creating toxicity or the recurrence of pain. Studies have shown that PCA provides pain relief superior to the traditional intramuscular route of administration, with fewer side effects and complications from narcotics and reduced nursing time for dose administration (Williams, 1992). By administering analgesic therapy with the patient's pain rhythm, then postoperative outcomes and recovery will be enhanced.

Nonpharmacologic approaches can often help divert attention from pain to alternate sensory experiences, which can further change the affective component of the pain experience. According to Sherwood, et. al. (2003), cognitive and behavioral nonpharmacological approaches have been associated with postoperative pain recovery and can be important approaches, particularly in short hospital stays. Patients in the past have reported high use of alternative approaches to pain relief such as prayer, massage, deep breathing, distraction, and repositioning (Sherwood, et. al., 2003). Acupuncture is another nonpharmacologic technique that is one of the oldest and most commonly used medical procedures across the globe. It originated in China more than 2,000 years ago and became better known in the United States in 1971, when New York Times reporter James Reston wrote about how doctors in China used needles to ease his pain after surgery (NCCAM, 2005). The report from a Consensus Development Conference on Acupuncture held at the National Institutes of Health (NIH) in 1997 stated that acupuncture is being widely practiced by many practitioners for relief or prevention of pain for various health conditions (NCCAM, 2005). It has been considered just as effective as analgesia. Many individuals have reported relief of various pain including back pain, gynecological pain from abdominal and pelvic surgeries, fibromyalgia, cancer pain, headache, etc.

According to the 2002 National Health Interview Survey it was estimated that 8.2 million U.S. adults had ever used acupuncture, and an estimated 2.1 million U.S. adults had used acupuncture in the previous year (NCCAM, 2005). The term acupuncture describes a family of procedures involving stimulation of anatomical points on the body by a variety of techniques (NCCAM, 2005). American practices of acupuncture have incorporated numerous medical traditions from China, Japan, Korea, and other countries. The acupuncture technique that has been most studied scientifically involves penetrating the skin with thin, solid, metallic needles that are manipulated by the hands or by electrical stimulation (NCCAM, 2005). Overall, acupuncture is a fairly safe intervention when practiced by regulated practitioners. The FDA approved acupuncture needles for use by licensed practitioners in 1996, and requires that sterile, nontoxic needles be used that they be labeled for single use by qualified practitioners only (NCCAM, 2005). Few complications have been reported to the FDA and most occurred from using inadequate sterilization of needles and from improper delivery of treatments.

Throughout the world, the medical profession has been showing an increased interest in acupuncture with the discovery of the possibility that it may trigger an endogenous opiate mechanism (Hobbs, 1994). Previous research has shown that electroacupuncture, the use of pulsed electric current to stimulate acupuncture needles, has a positive effect on post operative pain relief and decreased the need for narcotic requirement after abdominal operations in women (Christensen, P. A., et. al., 1989). Numerous research projects in the 1970s and the 1980s took place in China and in the Western hemisphere that have isolated a number of neurotransmitters that are involved in acupuncture. Researchers wanted to explore the precise role of endorphin and non-endorphin systems in acupuncture anaesthesia and the specific receptors involved (Hobbs, 1994). Such neurotransmitters include neuropeptides (opiate-like substances) such as endorphines, encephalines, serotonin (5HT), dynorphin A and B, substance P, as well as simple amino acids such as histamine, glycine, and glutamic acid, as well as catecholamines such as adrenalin and noradrenaline, and dopamine (Hobbs, 1994). Other neurotransmitters involved include acetylcholine, somatostatin, luteinizing hormone releasing hormone, bradykinin, prostoglandins, cyclic adenosine monophosphate and guanosine monophosphate, gamma-amino butyric acid, and thyrotropin-releasing hormone (Hobbs, 1994). According to Hobbs (1994), electroacupuncture analgesia induced by low frequency stimulation is mediated by endorphines, while high frequency is at least partly serotonergic.

Electro-acupuncture has also been used in numerous studies for treatment options. Among them there are studies associated with electro-acupuncture use for producing analgesia for operative interventions. According to Hobbs (1994), there was a study that looked to the effect of electroacupuncture on postoperative pain relief after lower abdominal/elective gynecological surgery in women. The study was designed to eliminate the need for a placebo control by using patient controlled analgesia for an objective assessment in pain (Hobbs, 1994). After surgery and while still anaesthetized, the patients received either electroacupuncture or no further treatment (Hobbs, 1994). It was found that the group receiving the electroacupuncture consumed half the quantity of pethidine as that used by the no-treatment group, in the first two hours following surgery (Hobbs, 1994). More common studies with acupuncture have been conducted with neck and back pain have received good results and patients have obtained a significant long-term improvement that is a relief from pain (Hobbs, 1994).

Assessment of pain is often measured on rating scales. This is called ordinal scaled data. After pain is assessed, then interventions can be provided. The McGill pain questionnaire (MPQ) is the most widely known and used verbal pain assessment tool. It is aimed to measure the multidimensional nature of pain by means of words used to describe the pain (Van Wijk & Hoogstraten, 2002). Following the Gate Control Theory of Pain, the MPQ was based on a theoretical model of pain processing in the brain and research on the language of pain (Van Wijk, et. al., 2002). MPQ was developed in such a way that both the intensity and the quality of pain could be measured and treated statistically, which made the MPQ very popular and a unique instrument in the area of pain research.

Transcutaneous electrical nerve stimulation (TENS) is a complementary medical technique that has been used for pain management in variety of medical conditions, such as chronic back pain, myofascial and arthritic pain, sympathetically mediated pain, bladder incontinence, neurogenic pain, visceral pain, post-surgical and dental procedure pain (Kaye & Murray, 2002).

The procedure involves passage of electric current through wires to electrodes pasted on skin. Electrodes are imbedded into skin pads, which may be placed throughout the body. Potential mechanisms of action include presynaptic inhibition in the dorsal horn of the spinal cord, endogenous pain control via endorphins, enkephalins, and dynorphins, restoration of afferent input, as well as direct inhibition on an abnormally excited nerve (Kaye & Murray, 2002). Electrical stimulation reduces pain through nociceptive inhibition at the presynaptic level in the dorsal horn, limiting its central transmission.

There is no use of needles in this procedure. There may be a variety of frequencies and intensities used during the stimulation session. There may be high frequency stimulation of 40-150 Hz and low intensity at 10-30 mA. The pulse duration is short, with up to 50 microseconds, and the onset of analgesia is immediate and short-lived (Kaye & Murray, 2002).

Percutaneous electrical nerve stimulation (PENS) has a technique that is superior in its delivery of stimulation to precise body points. It may also be referred to as electroacupuncture. It combines the benefits of both acupuncture and TENS (Kaye & Murray, 2002). It bypasses the resistance of the skin and delivers the electrical stimulation closer to the nerve endings in the soft tissues. Acupuncture needles or probes are placed at specific points in the body into the soft tissue or muscle, and pulsed electric current is applied to stimulate the area. Specific locations are determined by dermatome level consistent with pain location. It has been proposed that electroacupuncture analgesia induced by low frequency stimulation is mediated by endorphins, while high frequency stimulation is at least partly serotonergic (Hobbs, 1994).

Numerous studies on electroacupuncture are available in areas such as analgesia production for operative intervention, stroke treatment, nerve damage and addiction treatment (Hobbs, 1994). Specifically, it has been used for postoperative pain relief after lower abdominal surgery in women (Christensen, Noreng, Anderse & Neilsen, 1989), evidenced by reduction of pethidine used by subjects in the first 2 hours following surgery. In a later study in 1993, effects of electroacupuncture were again evaluated in anesthesia provision for hysterectomy patients (Christensen et al., 1993). This study was prospective, randomized and patient-blinded. The conclusion stated that no significant differences between control and experimental groups with respect to postoperative analgesic requirement, pain rating and metabolic stress responses were established. Potential reasons for absence of difference between the two groups were large amounts of induction and maintenance analgesic, short-lasting effects of pre and intra-operative use of electroacupuncture, as well as use of propofol instead of thiopentone as choice of analgesia. A more recent study on use of electroacupuncture for analgesia in major abdominal and pelvic surgery was a randomized, controlled trial of 250 cancer patients (Poulain, et al., 1997). There were statistically significant differences between electroacupuncture and TENS groups in use of opiates, but no variation in use of non-narcotic pain medications. Notably, patients in the PENS group were extubated in half the time, as compared to those in the TENS group, as well as required less analgesia.

PENS was studied as short-term management of headache. It was determined to be an effective complementary technique to analgesic and anti-migraine medications, unaffected by origin of headache symptoms (Ahmed et al., 2000). Low back pain is another condition that was researched in a randomized crossover study (Ghoname, et al., 1999). Results showed that PENS was more effective than TENS or exercise in short-term pain relief and improved physical function in patients with long term low back pain. Of particular interest is the use of controls in this study. Pain relief was compared in patients receiving PENS, PENS without electrical stimulation (“sham”), TENS and exercise. Use of PENS in treatment of long-term low back pain was continued by Weiner et al in 2003. This randomized, controlled clinical trial concluded that PENS may be a promising treatment modality for community-dwelling older adults with CLBP, as demonstrated by reduction in pain intensity and self-reported disability, and improvement in mood, life control, and physical performance (Weiner et al, 2003). Physical therapy, however, was used in both control PENS and experimental sham PENS groups.

### OBJECTS AND SUMMARY OF THE INVENTION

It is an object of the invention to provide a pain relief method that allows a patient to achieve the same result of pain relief as in a standard treatment but with a reduced amount of medications by combining a pharmacological treatment with percutaneous electrical nerve stimulation therapy or electro-acupuncture. It is an object of the present invention to provide a method of pain relief in women after abdominal/pelvic surgery by combining patient-controlled analgesia with percutaneous electrical nerve stimulation therapy or electro-acupuncture. It is another object to provide the aforementioned method wherein percutaneous electrical nerve stimulation or electro-acupuncture is carried out with the use of apparatuses that allow patients to have freedom of movement of carrying out the electrodes or needles over a long period of time and allow patients to regulate the intensity and duration of electrical stimulation according to their individual needs.

The method consists of reducing pain in a patient by combining a tradition pharmacological treatment with electro-acupuncture or percutaneous electrical nerve stimulation therapy. The pharmacological treatment consists of using analgesics such as morphine sulfate and hydromorphine provided to patients around the clock or as needed for pain. The patient controlled analgesia pump (PCA) is used by patients for self-administering of analgesia as needed. After patient stops using PCA pump narcotic and non-narcotic pain medications are administered mostly orally.

The method of the invention is intended for treating pain in women after abdominal/pelvic surgery and the purpose of treatment by combining patient-controlled analgesia with percutaneous electrical nerve stimulation or electro-acupuncture is to reduce the amount of pharmacological means required for achieving the same result of pain relief and thus to help patient to recover bowel function faster.

According to this method, post-surgical patient starts receiving percutaneous electrical nerve stimulation or electro-acupuncture after she comes out of general anesthesia, usually 24 hours after abdominal/pelvic surgery. Two pairs of needles/electrodes are inserted at an acute angle into points on the skin of a patient located at the medial aspect of the tibia, between the medial malleolus of the ankle and the inferior margin of the medial condyle of the tibia. Then, the external parts of the needles above the surface of skin are either attached to the patient's legs with the adhesive tape, or they are secured on the surface of the skin with a special electrode/needle holding devices which keep electrodes/needles attached to the legs in an inclined position. Electrical stimulator is then connected to the electrode needles via these holding devices or, in case of taped needles, via the adhesive pads that are connected to the outputs of the electrical stimulator on one end and to the exposed parts of the electrode needles that are not covered by tape on the other end. Each pair of electrodes is connected to the independent output of the electrical stimulator. Electrical stimulation is initiated by either a therapist or a patient by turning on the electrical stimulator. The intensity, duration, and the frequency modes are adjusted according to the patient's tolerance level and individual needs. Patient is able to switch stimulation on and off and to change the intensity and duration of stimulation as needed. The oblique orientation of the electrode needles in the soft tissues of the patient's leg and the ability of the electrodes, in accordance with this method, to stay on the body of the patient for long periods of time provides portability, freedom of movement, and lasting pain relief to the patient.

In a study researching the effectiveness of this method the author used the following protocol: There were three groups of patient with random assignment. First control group received only standard pharmaceutical treatment with PCA pump. Second control group received standard treatment along with non-electrical acupuncture used on the abovementioned points. Third group received standard care along with portable PENS. For patients that were assigned to receive acupuncture or electro-acupuncture/PENS, treatment was initiated 24 hours after the surgery. The needles were placed 24 hours following surgery and initiation of patient controlled analgesia. The PENS group received first session of electro-acupuncture 24 hours after the surgery. It lasted from 45 minutes to 1 hour and was administered by the licensed acupuncturist. Second session of the day of the same duration was administered 12 hours later. The needles stayed on the body throughout the day and were removed after the end of the 2nd session. The next day same sequence was repeated. Thus, each patient from the portable PENS group received 4 sessions in 48 hours.

Patients randomized to a non-electrical acupuncture group also had acupuncture treatment in the same points started in 24 hours post-surgically. They also had needles stay for the duration of the day. The needles were removed at the end of the day and the new acupuncture needles were inserted on the following day. Twice a day, each day, acupuncture needles were stimulated manually by the acupuncturist, once in the morning and once at night.

The modulation mode for electrical stimulation was utilized for this protocol. The frequency of the pulse repetition remained consistent, and pulse width changed cyclically. For example, if pulse width was set for the maximum output value (250 milisec), pulse width would decrease to 125 milisec and then increase again to 250 milisec in a period of 4 seconds.

To measure pain, McGill Pain Questionnaire and Visual Analog Pain Scale were given to patients 1 hour before and 1 hour after each treatment (4× daily each group). Medication usage and pain were monitored through St. Elizabeth's Caritas Nursing Postoperative Flow chart×2 days.

According to the midway results (it is an ongoing study) with 10 people in each group having completed the protocol at the time of writing, patients from portable PENS group experienced 50% greater reduction in pain in average during the electro-stimulation than patients from acupuncture group during non-electrical acupuncture as measured by Visual Analog Pain Scale before and after each session. Eight out of ten PENS patients discontinued PCA pump after first PENS session.

### BRIEF DESCRIPTION OF THE DRAWINGS

FIG. 1 is a view of a leg with preferred position of points for insertion of needle electrodes according to the method of the invention.

FIG. 2 is a schematic view of the entire electro-acupuncture apparatus used for realization of the method of the invention.

FIG. 3 is view that shows an assembly of a needle electrode with a needle holding device and positions of points of needle insertion.

### DETAILED DESCRIPTION OF THE INVENTION

While this method is well-suited to treat low abdominal/pelvic pain of various etiologies, it is particularly effective in postoperative pain control for women undergoing gynecological or abdominal surgery.

According to this method, post-surgical patient starts receiving percutaneous electrical nerve stimulation or electro-acupuncture after she comes out of general anesthesia

According to one of the implementations of this method, treatment starts post-surgically, usually 24 hours after patient comes out of general anesthesia. This time is given as an example and the treatment can be started earlier or later than 24 hours. The percutaneous electrical nerve stimulation is administered to the points located on the medial side of the low extremities, near the posterior margin of tibia, where saphenous nerve passes. In its basic form, there can be only one needle and one standard electrode for subcutaneous electrical nerve stimulation used on one leg only. In this case, standard acupuncture needle is inserted at an acute angle into the point located between the medial condyle of the tibia and medial malleolus of the leg. The other electrode with the adhesive layer is attached at a different place on the same trajectory above the saphenous nerve next to the medial side of the tibia, between the medial condyle of the tibia and the medial malleolus of the leg.

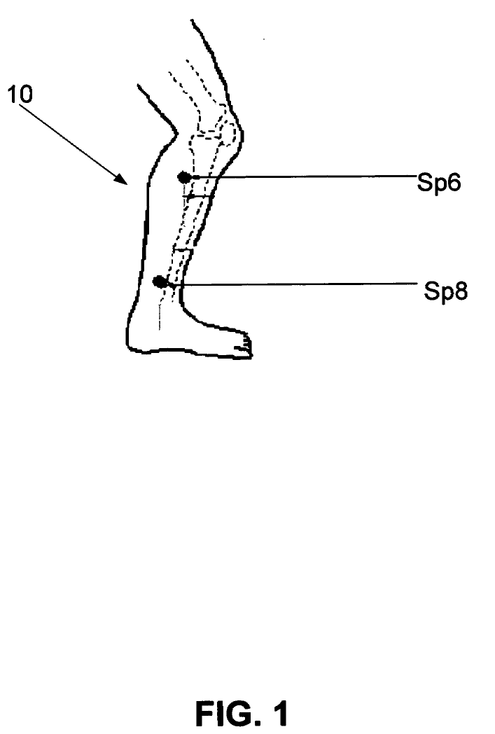

In a preferred implementation, therapist inserts four standard acupuncture needles/electrodes at the acute angles into four points, two on each leg. FIG. 1 is a view of a leg 10 with preferred position of points Sp6 and Sp8 for insertion of needle electrodes according to the method of the invention. Designations Sp6 and Sp8 corresponds to the standard acupuncture point nomenclature. Sp6 is located at the posterior margin of the tibia, 3 units above the medial malleolus of the ankle (ankle bone). Sp8 is located at the posterior margin of the tibia 3 units below the inferior margin of the medial condyle of the tibia. There are 13 units between medial condyle of the tibia and medial malleolus.

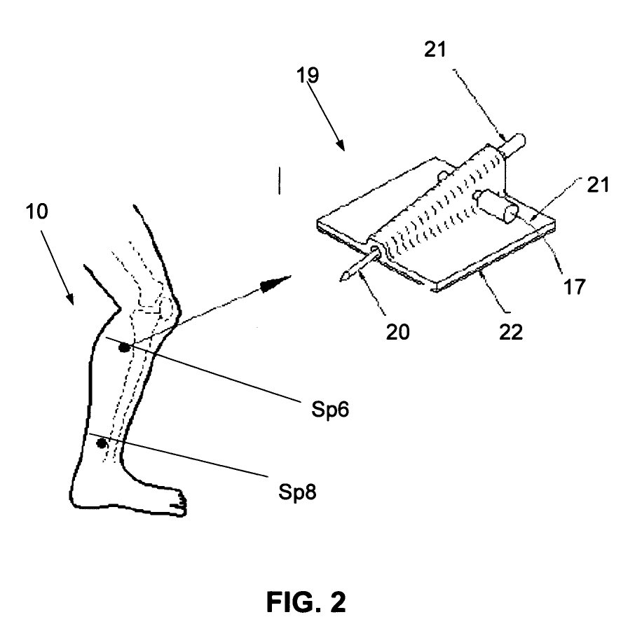

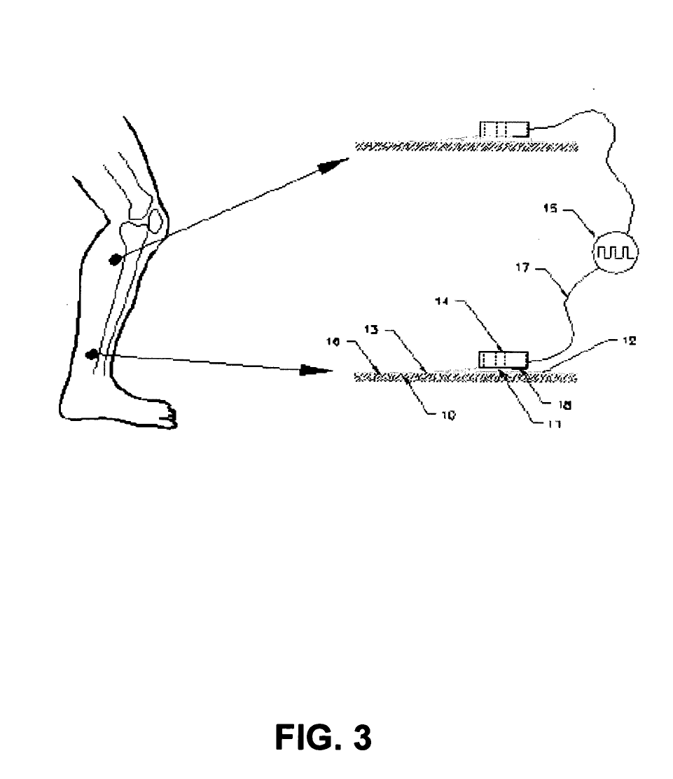

The needles are secured in the above mentioned points on the patient's legs either with a help of a special holding device, FIG. 3, described in more detail in pending U.S. patent application Ser. No. 11/078,765, or with the help of adhesive insulating tape and adhesive electrodes, FIG. 2, described in pending U.S. patent application Ser. No. 10/962,299, wherein FIG. 2 is a schematic view of the entire electro-acupuncture apparatus used for realization of the method of the invention, and FIG. 3 is view that shows an assembly of a needle electrode with a needle holding device and positions of points of needle insertion.

A needle electrode holding shown in FIG. 3 is designated as a whole by reference numeral 19. Two such devices 19 are first attached to the skin at the Sp6 and Sp8 locations. Since both needle electrode holding devices 19 are identical, only one of them shown in FIG. 3 will be described. The needle electrode holding device 19 is attached to the patient's skin in the aforementioned points via an adhesive layer 22. The device 19 is attached precisely so that the tip of the needle 20 stays just above the corresponding point when the needle is loaded into the device. Practitioner inserts the needle 20 by pushing on a needle handle 21, while the needle remains in the inclined position to the skin inside the device. Electrical wires 17 provides electrical connection between the needles and the electrical simulator 15.

In the most basic version, practitioner first inserts the needle 20 into the corresponding point, then, separates the external part of the needle from the skin 16 (FIG. 2) by attaching insulating adhesive tape 12 under the needle to the skin. The second piece of the tape 13 insulates the point of insertion and the external part of the needle on top, and is glued to the lower tape. Thus, the needle 20 is “sandwiched” between two pieces of tape 12 and 13 and is attached to the skin 16. There is a portion of the handle 21 which remains exposed. Adhesive electrode 14, which is then placed above the second tape, adhesive conductive layer first, gets in contact with the handle 21 along the exposed part thereof. Electrical wire 17 connects each needle/electrode assembly 19 to the electrical stimulator 15 (FIG. 3).

After all the needles get safely secured against the skin on the medial aspect of the tibia in the inclined positions through the described procedure and apparatuses, percutaneous electrical nerve stimulation is initiated. Typically, there are two pairs of needle electrodes 20, two on each leg, connected to two separate outputs of the portable electrical stimulator 15. The intensity of the stimulation is gradually increased to the level that is well tolerated by a patient, yet, is clear and distinct. The modulation mode of electrical stimulation is preferable since there is less habituation to it. Sensation tends to diminish with time and the output has to be increased to reach the same level of electrical sensation. Patient is instructed how to control the stimulator 15.

Each session should last at least 30 minutes to 1 hour, or longer, for better results. Multiple sessions in a course of one day are possible and advisable if the pain is persistent and severe. In general, after abdominal/pelvic surgery, patients are encouraged to start moving as soon as possible. Movement helps patients to regain intestinal motility and normal bowel function and avoid adhesions. Due to the portability of the devices, this method allows freedom of movement while providing pain relief. This method not only helps with the bowel function indirectly, through lowering narcotic medications that tend to decrease intestinal motility, but it also promotes peristalsis through the percutaneous electrical stimulation at points Sp6 and Sp8. In the acupuncture practice these points are often used to promote peristalsis. The patient is instructed to adjust the level of stimulation according to her tolerance level and the time, when stimulation is used, according to the patient's specific pain pattern. The immediate response to pain with stimulation is both very effective and psychologically comforting to the patient.

The needles 20 and all holding assemblies 19 are removed by the end of the day to give the skin and the tissues time to restore. If needed, the procedure is repeated next day.

The experiments conducted by the inventor showed that when electrical stimulation was applied to the needles according to the described method, the reduction in pain was at least 50% greater than with regular acupuncture performed on the same points for the same period of time. Significant reduction in pain was observed in just 45 minutes after administering electrical stimulation.

Thus, it has been shown that the invention provides a pain relief method that allows a patient to achieve the same result of pain relief as in a standard treatment but with a reduced amount of medications by combining a pharmacological treatment with percutaneous electrical nerve stimulation therapy or electro-acupuncture. The method is intended for treating women after abdominal/pelvic surgery by combining patient-controlled analgesia with percutaneous electrical nerve stimulation therapy or electro-acupuncture. In the proposed method, percutaneous electrical nerve stimulation or electro-acupuncture is carried out with the use of apparatuses that allow patients to have freedom of movement of carrying out the electrodes or needles over a long period of time and allow patients to regulate the intensity and duration of electrical stimulation according to their individual needs.

Although the invention has been shown and described with reference to specific embodiments, it is understood that these embodiments should not be construed as limiting the areas of application of the invention and that any changes and modifications are possible, provided these changes and modifications do not depart from the scope of the attached patent claims. For example, the needle electrodes can be inserted not necessarily in points Sp6 and Sp8 but in any location on the line between the medial malleolus of the ankle and the inferior margin of the medial condyle of the tibia. Although two needles were mentioned in the description, at least one needle or more than two needles can be used. In addition to treatment of pain in patients after abdominal/pelvic or gynecologic surgery, the method of the invention may be used for treating other conditions, e.g., for improving peristalsis of the intestines, reducing nausea, preventing formation of the adhesions, increasing sense of well-being, reducing hospital stay, etc. It can be used in the hospital, at home, or other safe locations. The amount of medications that are used at the beginning along with percutaneous nerve stimulation can be reduced to minimal or none at all if the pain can be controlled only with percutaneous electrical nerve stimulation.

1. A method of pain relief in a patient after abdominal/pelvic or gynecologic surgery comprising the steps of combining pharmacological treatment with percutaneous electrical nerve stimulation, said percutaneous electrical nerve stimulation being carried out with the use of needle electrodes of the type that allows the patient to have freedom of movement so that the patient can carry the needle electrodes for the entire day, if needed.

2. The method of claim 1, comprising the steps of: providing at least one said needle electrode and an electrical stimulator connected to the aforementioned needle electrode; conducting the aforementioned pharmacological treatment and conducting the percutaneous electrical nerve stimulation by administering the aforementioned electrode in a point located on the medial side of a low extremity of the patient near the posterior margin of tibia where saphenous nerve passes.

3. The method of claim 2, wherein the aforementioned pharmacological treatment is a patient-controlled analgesia.

4. The method of claim 2, wherein said percutaneous electrical nerve stimulation comprises the steps of:
inserting said at least one needle electrode at an acute angle into a point on the skin of a patient located at the medial aspect of the tibia, between the medial malleolus of the ankle and the inferior margin of the medial condyle of the tibia;
attaching the needle electrode to the patient's leg with the attachment means connecting the needle electrode to the electrical stimulator; and
electrically stimulating the aforementioned point.

5. The method of claim 3, wherein said percutaneous electrical nerve stimulation comprises the steps of:
inserting said at least one needle electrode at an acute angle into a point on the skin of a patient located at the medial aspect of the tibia, between the medial malleolus of the ankle and the inferior margin of the medial condyle of the tibia;
attaching the needle electrode to the patient's leg with the attachment means connecting the needle electrode to the electrical stimulator; and
electrically stimulating the aforementioned point.

6. The method of claim 1, comprising the steps of:
providing a first needle electrode, a second needle electrode, and an electrical stimulator connected to the aforementioned first needle electrode and second needle electrode;
conducting the aforementioned pharmacological treatment;
administering the first needle electrode at an acute angle into a first point on the skin of a patient located at the medial aspect of the tibia at least on one patient's leg, between the medial malleolus of the ankle and the inferior margin of the medial condyle of the tibia;
attaching said first needle electrode to the patient's skin;
connecting said first needle electrode to the electrical stimulator;
administering the second needle electrode at an acute angle into a second point which is different from the first point on the skin of a patient located at the medial aspect of the tibia, between the medial malleolus of the ankle and the inferior margin of the medial condyle of the tibia;
connecting the second needle electrode to the electrical stimulator; and
electrically stimulating the first point and the second point.

7. The method of claim 6, wherein the aforementioned pharmacological treatment is a patient-controlled analgesia.

8. The method of claim 6, wherein the first point is the Sp6 point and the second point is the Sp8 point, according to the acupuncture nomenclature.

9. The method of claim 7, wherein the first point is the Sp6 point and the second point is the Sp8 point, according to the acupuncture nomenclature.

10. The method of claim 6, wherein the first needle electrode and the second needle electrode are held in needle holders that allow the patient to have freedom of movements so that the patient can carry the needle electrodes for the entire day, if needed.

11. The method of claim 7, wherein the first needle electrode and the second needle electrode are held in needle holders that allow the patient to have freedom of movements so that the patient can carry the needle electrodes for the entire day, if needed.

12. The method of claim 8, wherein the first needle electrode and the second needle electrode are held in needle holders that allow the patient to have freedom of movements so that the patient can carry the needle electrodes for the entire day, if needed.

13. The method of claim 9, wherein the first needle electrode and the second needle electrode are held in needle holders that allow the patient to have freedom of movements so that the patient can carry the needle electrodes for the entire day, if needed.

14. The method of claim 6, where a pair of needle electrodes is used on each patient's leg.

15. The method of claim 7, where a pair of needle electrodes is used on each patient's leg.

16. The method of claim 8, where a pair of needle electrodes is used on each patient's leg.

17. The method of claim 9, where a pair of needle electrodes is used on each patient's leg.

18. The method of claim 1, wherein the percutaneous electrical nerve stimulation is carried out at least in one session that lasts at least 30 minutes to 1 hour.

19. The method of claim 6, wherein the percutaneous electrical nerve stimulation is carried out at least in one session that lasts at least 30 minutes to 1 hour.

20. The method of claim 17, wherein the percutaneous electrical nerve stimulation is carried out at least in one session that lasts at least 30 minutes to 1 hour.
